(19) United States
(12) Patent Application Publication
(10) Pub. No.: US 2025/0387038 A1
(43) Pub. Date: Dec. 25, 2025

(54) PRECISION PHYSIOLOGICAL SENSOR

(71) Applicant: Stellar Wave Technologies, Inc., Los Altos, CA (US)

(72) Inventor: Stephen Linsay Francis Lloyd, Los Altos, CA (US)

(21) Appl. No.: 19/242,669

(22) Filed: Jun. 18, 2025

Related U.S. Application Data
(60) Provisional application No. 63/661,847, filed on Jun. 19, 2024.
(60) Provisional application No. 63/781,766, filed on Apr. 1, 2025.

(51) Int. Cl. A61B 5/05; A61B 5/00; A61B 5/0205; G16H 40/67

(52) U.S. Cl. CPC A61B 5/05; A61B 5/0205; A61B 5/681; A61B 5/6898; A61B 5/7203; A61B 5/7225; A61B 5/725; G16H 40/67

(57) ABSTRACT

A sensor system is described. The system includes a signal generator, a transmitter subsystem, and a receiver subsystem. The signal generator is configured to provide a radio frequency (RF) signal having a first frequency. The transmitter subsystem is coupled to the signal generator. The transmitter subsystem is configured to transmit an output signal based on the RF signal. The output signal has a second frequency greater than the first frequency. The receiver subsystem is coupled to the signal generator. The receiver subsystem is configured to detect a reflected signal from a target based on a first detection signal and a second detection signal. The first detection signal and the second detection signal are based on the RF signal.

### CROSS REFERENCE TO OTHER APPLICATIONS

This application claims priority to U.S. Provisional Patent Application No. 63/661,847 entitled PRECISION DETECTOR filed Jun. 19, 2024, and U.S. Provisional Patent Application No. 63/781,766 entitled PRECISION PHYSIOLOGICAL SENSOR filed Apr. 1, 2025, both of which are incorporated herein by reference for all purposes.

### BACKGROUND OF THE INVENTION

Detection of various physiological parameters is desired. For example, heart rate, variation in heart rate, temperature, other skin characteristics that might be imaged, blood vessels proximate to the skin, changes in such blood vessels, and/or other physiological properties might be detected. Currently, sensors that are in physical contact with the skin are capable of detecting at least some of these parameters. However, such sensors are typically bulky, may require continuous physical contact over a longer period of time, or have other limitations.

Radio frequency (RF) systems may be used for imaging and detection of other physiological parameters such as temperature. Such RF systems may be considered herein to include millimeter wave systems (e.g., approximately 30 GHz to 300 GHz) and/or microwave systems (e.g. approximately 300 MHz to 30GHz). However, such RF systems may have significant drawbacks. In some cases, such RF systems utilize acoustics or other techniques for detection. Such systems may be bulky or provide insufficient sensitivity for imaging or detection of some physiological parameters. Other RF systems may face significant limitations that can impede system performance and efficiency. For example, such RF systems may be susceptible to image frequency interference or other sources of noise that adversely impact signal detection. Thus, such RF systems may have poor performance and/or be bulky. Consequently, improved techniques for detecting physiological parameters are desired.

### BRIEF DESCRIPTION OF THE DRAWINGS

Various embodiments of the invention are disclosed in the following detailed description and the accompanying drawings.

FIG. 1 is a block diagram of an embodiment of a radio frequency sensor system.

FIG. 2 is a diagram of an embodiment of a radio frequency sensor system.

FIG. 3 is a diagram of an embodiment of a radio frequency sensor system.

FIG. 4 is a diagram of an embodiment of a radio frequency sensor system.

FIG. 5 is a diagram of an embodiment of a radio frequency sensor system.

FIG. 6 is a flow-chart depicting an embodiment of a method for sensing using a radio frequency sensor system.

FIG. 7 is a flow-chart depicting an embodiment of a method for sensing using a radio frequency sensor system.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

A sensor system is described. The system includes a signal generator, a transmitter subsystem, and a receiver subsystem. The signal generator is configured to provide a radio frequency (RF) signal having a first frequency. The transmitter subsystem is coupled to the signal generator. The transmitter subsystem is configured to transmit an output signal based on the RF signal. The output signal has a second frequency greater than the first frequency. The receiver subsystem is coupled to the signal generator. The receiver subsystem is configured to detect a reflected signal from a target based on a first detection signal and a second detection signal. The first detection signal and the second detection signal are based on the RF signal.

In some embodiments, the sensor system further includes a splitter coupled to the signal generator, the transmitter subsystem, and the receiver subsystem. The splitter splits the RF signal into multiple signals including a first RF signal input to the transmitter subsystem, a second RF signal, and a third RF signal. The second RF signal and the third RF signal are input to the receiver subsystem. The second RF signal corresponds to the first detection signal. The third RF signal corresponds to the second detection signal.

In some embodiments, the transmitter subsystem includes at least one frequency multiplier coupled with the splitter. The frequency multiplier is configured to multiply the first frequency of the first RF signal to provide the second frequency of the output signal. In some embodiments the receiver subsystem further includes a multiplier and a first mixer. The multiplier is coupled with the splitter and configured to multiply the first frequency of the second RF signal for the first detection signal. The first detection signal has a first detection signal frequency. The first mixer is coupled with the multiplier and configured to mix the reflected signal with the first detection signal to provide an intermediate frequency signal having an intermediate frequency. In some such embodiments, the receiver subsystem further includes a phase splitter and a second mixer. The phase splitter receives the third RF signal and outputting the second detection signal. The second mixer is coupled with the phase splitter and the first mixer. The second mixer combines at least a portion of the intermediate frequency signal and the second detection signal. The second mixer outputs a detection signal.

In some embodiments, the receiver subsystem further includes an analog-to-digital (ADC) converter coupled with the second mixer. The ADC converter provides a digital output signal based on the detection signal. The receiver subsystem may further include an amplifier and a band pass filter. The amplifier is coupled with the first mixer. The band pass filter coupled with the amplifier and the second mixer, the band pass filter configured to pass a frequency range including the intermediate frequency.

In some embodiments, the sensor system is configured to detect, based on the reflected signal, at least one of heart rate, heart rate variation, human skin, temperature, skin characteristics, blood vessels, or a change in blood vessels.

A wearable system including a sensor subsystem and an attachment subsystem is described. The sensor subsystem includes a signal generator, a transmitter subsystem coupled to the signal generator, and a receiver subsystem coupled to the signal generator. The signal generator is configured to provide a radio frequency (RF) signal having a first frequency. The transmitter subsystem is configured to transmit an output signal based on the RF signal. The output signal has a second frequency greater than the first frequency. The receiver subsystem is configured to detect a reflected signal from a target based on a first detection signal and a second detection signal. The first detection signal and the second detection signal are based on the RF signal. The attachment subsystem is configured to couple the sensor subsystem to a user.

In some embodiments, the sensor subsystem of the wearable system further includes a splitter coupled to the signal generator, the transmitter subsystem, and the receiver subsystem. The splitter splits the RF signal into a plurality of signals including a first RF signal input to the transmitter subsystem, a second RF signal, and a third RF signal. The second RF signal and the third RF signal are input to the receiver subsystem. The second RF signal corresponds to the first detection signal. The third RF signal corresponds to the second detection signal. In some embodiments, the wearable system is incorporated into at least one of a watch or a smart phone. In some embodiments, the attachment subsystem includes at least one of a wrist band or a chest band.

A method is described. The method includes transmitting, by a transmitter subsystem coupled to a signal generator that generates a radio frequency (RF) signal having a first frequency, an output signal. The output signal is based on the RF signal. The output signal has a second frequency greater than the first frequency. The method also includes detecting, by a receiver subsystem, a reflected signal from a target. The detecting is based on a first detection signal and a second detection signal. The first detection signal and the second detection signal are based on the RF signal.

In some embodiments, the method also includes splitting the RF signal into a plurality of signals including a first RF signal input to the transmitter subsystem, a second RF signal, and a third RF signal. The second RF signal and the third RF signal are input to the receiver subsystem. The second RF signal corresponds to the first detection signal. The third RF signal corresponds to the second detection signal. In some embodiments, the transmitting further includes multiplying the first frequency of the first RF signal to provide the second frequency of the output signal.

In some embodiments, the receiving further multiplying the first frequency of the second RF signal for the first detection signal. The first detection signal has a first detection signal frequency. The receiving also includes mixing the reflected signal with the first detection signal to provide an intermediate frequency signal. The first intermediate frequency signal has an intermediate frequency. The receiving may further include receiving the third RF signal at a phase splitter and outputting, by the phase splitter, the second detection signal. The receiving also includes mixing at least a portion of the intermediate frequency signal and the second detection signal. Thus, a detection signal is provided. In some embodiments, the receiving further includes analog-to-digital (ADC) converting the detection signal to provide a digital output signal.

The receiving may also include amplifying the intermediate frequency signal to provide an amplified intermediate. The receiving may also include filtering the amplified intermediate frequency signal to pass a frequency range including the intermediate frequency. Thus, the at least the portion of the intermediate frequency signal is provided for mixing with the second detection signal. In some embodiments, the receiver subsystem is configured to detect, based on the reflected signal, at least one of heart rate, heart rate variation, human skin, temperature, skin characteristics, blood vessels, or a change in blood vessels.

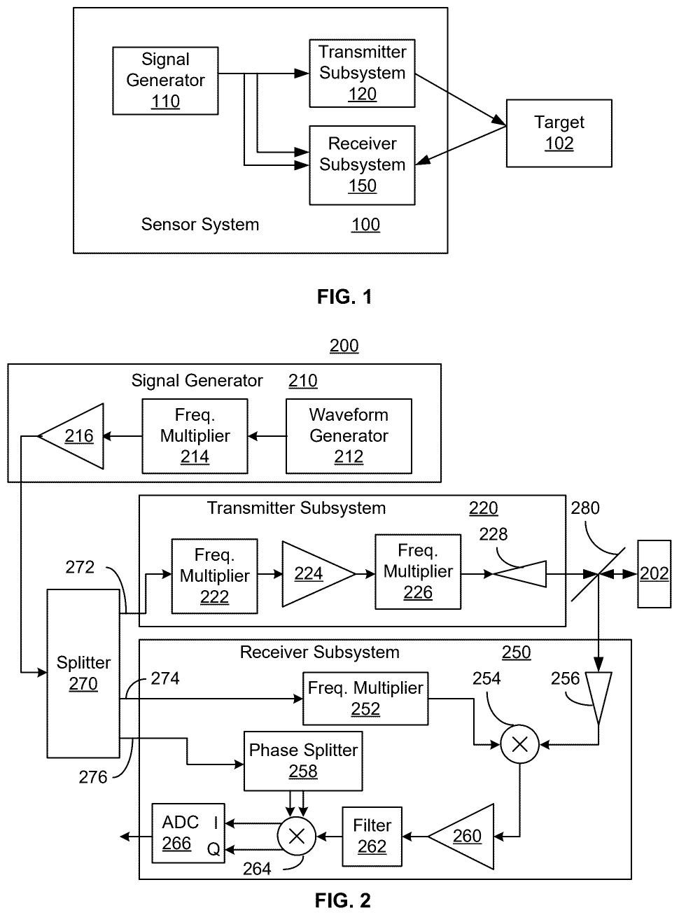

FIG. 1 is a block diagram of an embodiment of sensor system 100. Sensor system 100 includes radio frequency (RF) signal generator 110, transmitter subsystem 120, and receiver subsystem 150. Sensor system 100 is used to interrogate target 102. In some embodiments, sensor system 100 is an RF sensor system. For simplicity, sensor system 100 is thus described as an RF sensor system. RF sensor system 100 interacts with target 102 (e.g., a human/human skin). In some such embodiments, RF signals in the sub-terahertz range (e.g., up to 700 GHz) may be used. In some embodiments, the RF signals for RF sensor subsystem may have frequencies in the range of 30 kHz to 700 GHz (e.g., using millimeter waves in the approximately 30 GHz to 300 GHz frequency range and/or microwave radiation in the approximately 300 MHz to 30GHz range). In some embodiments, the signals may also be used for communication between devices. Further, such signals may be used for wireless charging between devices at distance capability using the same frequency band and antennas.

RF sensor system 100 includes signal generator 110, transmitter subsystem 120, and receiver subsystem 150. In some embodiments, RF sensor system 100 is part of a sensor array. For example, RF sensor system 100 may be part of a system on a chip (SOC) that includes multiple sensor systems 100 or multiple portions of sensor system 100. For example, such a sensor array may include multiple transmitter subsystems 120 and/or multiple receiver subsystems 150 and one or more signal generators 110. RF sensor system 100 may be combined with other sensing systems (not shown) to provide a wider variation in sensing capabilities. For example, RF sensor system 100 may be combined with acoustic, optical, or other measurement techniques. RF sensor system 100 may be mounted on or part of a smart phone, a smart watch, a bracelet, or necklace, a tablet computer, or other device that may be brought in close proximity to the target of interest. Thus, RF sensor system 100 may be part of a wearable or other portable device. In some such embodiments, RF sensor system 100 may be used in an array to provide a virtual array of pixels. In some embodiments, RF sensor system 100 may be used to continuously measure physiological characteristics of a person or other subject.

RF sensor system 100 may be in devices other than wearables. For example, RF sensor system 100 may be built into household items such as mirrors, chairs, and beds, automobiles, or computer monitors and laptop screens. Such RF sensor systems 100 may be used in conjunction with sensor(s) in wearables (including other RF sensor subsystem(s) 100 in the wearable(s)). Such sensors may form an ecosystem such that measurements from several sensors can be combined for both improved accuracy and true continuous monitoring. In some embodiments, RF sensor systems 100 in other devices may be used in the absence of sensor(s) in wearables.

If incorporated into wearables or other portable devices, remote wireless charging of RF sensor system 100 may be provided using multiple fixed sensors in household items, automobiles, or computers such that wearable sensors do not need to be removed for charging. This ecosystem of sensors may allow data transfer from RF sensor system 100 in one device to other devices and/or from the other devices to the device. RF sensor system 100 may combine this information with data from fixed sensors. The fixed sensor may perform measurements on the same person. The combined information may be sent to the cloud for processing, e.g. with a large artificial intelligence/machine learning model.

If incorporated into a wearable, RF sensor system 100 may use low power radios such as Bluetooth (BT) or Bluetooth low energy (BLE), or high bandwidth sub-terahertz radio for data transfer to the fixed sensors. High power fixed sensor radios, which may support wireless charging, may be used to provide thermal acoustic impulses on the target and measure additional diagnostic health information.

RF sensor system 100 may have a variety of modes. For example, RF sensor system 100 may be used as a radiometer, a spectrometer, radar, a temperature sensor, an image sensor, or other analogous sensor. In some embodiments, RF sensor system 100 may be used to collect physiological data (e.g. medical diagnostic vital signs of a person). For example, RF sensor system 100 may be utilized in measuring respiration rate, heart rate, the presence/absence of skin (e.g. human skin), variation in heart rate, vibrations in the skin of a subject, heart sounds (e.g., S1 and S2 (opening/closing of the heart valve), S3 and S4 (other sounds that may indicate underlying conditions)), SCG (seismocardiogram), BCG (ballistocardiogram), temperature and/or thermography, imaging or otherwise measuring skin characteristics (including up to a few centimeters under the skin) including sweat ducts, and other microstructures such as blood vessels. In addition various physiological characteristics, RF sensor system 100 may detect changes in such characteristics. For example, RF sensor system 100 may be used in detecting blood vessels and/or changes in blood vessels (e.g. variations in size or placement that may be due to temperature or other changes).

When in radar mode, multiple RF sensor systems 100 may be used in mono-static mode (every RF transceiver operating independently) or in a multi-static mode (transmitted signal from one RF sensor system 100 is also detected by another RF sensor system 100 in receive mode). The use of multi-static mode may better manage transmit-to-receive leakage (which is generally much stronger in the monostatic mode) and to allow the creation of better resolution (e.g., more pixels in the virtual array) as well as wider field of view (FOV). Signal processing and fusion of multiple RF sensor systems 100 which measure respiration and heart rate may be performed. Thin data may be used to improve the accuracy of chest (or other anatomies like wrist) vibration measurements to more accurately detect small vibrations of the lung, heart, and/or blood vessels. Thus, sensor system 100 may have utility in detecting a variety of physiological characteristics.

RF sensor system may be in contact with target 102 or may be spaced apart from target 102 by a small distance (e.g. at least a few micrometers, at least one millimeter, at least one centimeter, at least five centimeters, at least ten centimeters, and not more than two feet) during at least part of the time RF sensor system 100 operates. In some cases, the distance between RF sensor system 100 and target 102 may change during operation.

Signal generator 110 provides an RF signal having a first frequency. For example, the frequency of the RF signal may be in the range of 10 GHz through 30 GHz in some embodiments. Other frequencies are possible. Signal generator 110 is coupled to and provides the RF signal to both transmitter subsystem 120 and receiver subsystem 150. More specifically, signal generator 110 provides one RF signal to transmitter subsystem 120 and two RF signals to receiver subsystem 150. Thus, both transmitter subsystem 120 and receiver subsystem 150 utilize the RF signal from signal generator 110 during operation.

Transmitter subsystem 120 is configured to transmit an output RF signal based on the RF signal from signal generator 110. In some embodiments, transmitter subsystem 120 up converts the frequency (i.e., increases the frequency of) the RF signal. Transmitter subsystem 120 may also amplify the RF signal to provide an output RF signal. Thus, the output signal provided (e.g., radiated) to target 102 has a second frequency greater than the first frequency of the RF signal from signal generator 110. In some embodiments, transmitter subsystem 120 also controls other aspects of the output signal. For example, in some embodiments, transmitter subsystem 120 controls the polarization of the output signal.

Receiver subsystem 150 utilizes two copies of the RF signal in detecting the reflected signal from target 102. Receiver subsystem 150 down converts and detects this reflected signal from target 102 using a first detection signal and a second detection signal. The first and second detection signals are both based on the RF signal. Thus, the RF signals from signal generator 110 are processed and used in detecting the reflected signal from target 102. For example, one RF signal from signal generator 110 may have the first frequency up converted to form the first detection signal. Another RF signal from signal generator 110 may be split into two phases or otherwise processed to obtain the second detection signal used by receiver subsystem 150. In some embodiments, receiver subsystem 150 filters the reflected signal from target 102. For example, if the output signal from transmitter subsystem 120 has a first polarization, then receiver subsystem 150 may filter input signals such that only signals having a second polarization that is the reflection of the first polarization are accepted. In one such embodiment, if the output signal is left circularly polarized, then receiver subsystem 150 utilizes a filter that transmits right circularly polarized light. Other analogous techniques for filtering signals may be used.

RF sensor system 100 may have improved performance. In some embodiments, transmitter subsystem 120 may utilize low power, smaller, and lower cost components for generating the output signal. For example, the components used for up converting the RF signal from signal generator 110 may reduce the size, power consumption and cost of transmitter subsystem. Use of the RF signal from RF generator 110 for both up conversion in transmitter subsystem 120 and down conversion and detection in receiver subsystem 150 may reduce noise and increase sensitivity. Thus, performance, cost, and/or size of RF sensor system 100 may be improved.

FIG. 2 is a diagram of an embodiment of RF sensor system 200. RF sensor system 200 is analogous to RF sensor system 100. RF sensor system 200 thus may include similar components and/or have similar functions to RF sensor system 100. For example, RF sensor system 200 may be used in wearables and/or other devices, may measure physiological characteristics (e.g., heart rate, temperature, imaging etc.), and may share the benefits of RF sensor system 100. RF sensor system 200 thus includes signal generator 210, transmitter subsystem 220, and receiver subsystem 250 that are analogous to signal generator 110, transmitter subsystem 120, and receiver subsystem 150, respectively. RF sensor system 200 also includes splitter 270 and screen 280.

Signal generator 210 includes waveform generator 212, frequency multiplier 214 and amplifier 216. Waveform generator 212 may be an arbitrary waveform generator (AWG) that can directly generate a signal at a desired frequency. The waveform generator 212 may also provide chirp modulation. Frequency multiplier 214 multiplies the frequency of the signal to provide the desired RF frequency (the first frequency described with respect to signal generator 110). Frequency multiplier 214 may be subject to power attenuation in its output. Amplifier 216 may be used to provide the desired power for the RF signal. Thus, signal generator 210 may provide an RF signal having the desired power and the desired (first) frequency.

Splitter 270 is a three-way power splitter that separates the RF signal into three paths 272, 274, and 276. Each path carries an RF signal having the first frequency. First path 272 is coupled to and provides a first RF signal to transmitter subsystem 220. Second path 274 and third path 276 are coupled to and provide second and third RF signals, respectively, to receiver subsystem 250. Thus, first, second, and third RF signals having the first frequency are provided to transmitter subsystem 220 on path 272 and to receiver subsystem 250 on second path 274 and third path 276.

Transmitter subsystem 220 includes frequency multipliers 222 and 226, amplifier 224, and antenna 228. Frequency multiplier 222 multiplies the frequency (i.e., up converts the frequency) of the first RF signal by a specified amount. Amplifier 224 amplifies this signal, which may account for attenuation in processing of the signal. Frequency multiplier 226 multiplies the frequency of the upconverted, amplified RF signal by another amount. For example, frequency multipliers 222 and 226 may each multiply the frequency of the first RF signal by an integer. The multiplication may be by the same or different integers. Thus, the output of frequency multiplier is an RF signal having a higher (e.g. second) frequency greater than the frequency of the first RF signal input to transmitter subsystem 220.

Antenna 228 radiates the output RF signal. In some embodiments, antenna 228 radiates signals having a particular polarization, such as right-handed circularly polarized (RHCP) signals. In some embodiments, antenna 228 radiates the signal as a focused beam. For example, antenna 228 may have a parabolic shape to focus the radiation to a smaller spot or beam. Antenna 228 may be mounted onto mechanical gimbal or other apparatus to allow the spot to be swept across target 202 (e.g. in a 2-dimensional pattern). Because the radar provides the z-axis distance from antenna 228, a 3-dimensional (x-y-z) surface may be measured.

In the embodiment shown, screen 280 transmits RCHP signals, but reflects left-handed circularly polarized (LHCP) signals. Thus, screen 280 allows the RF output signal of transmitter subsystem 220 to be provided to target 202. Target 202 may be or include a portion of a human body (e.g. skin). Target 202 reflects the RF signals back to screen 280. However, such reflected signals are LCHP signals. Thus, screen 280 reflects such signals to receiver subsystem 250.

Receiver subsystem 250 includes frequency multiplier 252, mixer 254, antenna 256, phase splitter 258, amplifier 260, filter 262, mixer 264, and analog to digital (ADC) converter 266. Antenna 256 is an LHCP antenna. Antenna 256 thus has high gain for LHCP signals and very low gain for the RHCP signals, which may radiate from transmitter subsystem 220 into antenna 256. Thus, antenna 256 receives LHCP signals reflected from screen 280 (and thus including RF signals reflected from target 202). In some embodiments, antenna 256 may be mounted in a manner analogous to antenna 228. Thus, antenna 256 may also sweep across target 202. Further, antenna 256 may have a parabolic shape that may receive and/or focus reflected LHCP signals from screen 280.

The second RF signal having the first frequency is received from splitter 270 at receiver subsystem 250 on path 274. This second RF signal is provided to frequency multiplier 252, which up converts (e.g. multiplies) the frequency of the second RF signal. This higher frequency RF detection signal is provided from frequency multiplier 252 to mixer 254. Mixer 254 combines the higher frequency RF detection signal from frequency multiplier 252 with the reflected LHCP RF signal from antenna 256. In some embodiments, mixer 254 is a sub-harmonic mixer. Thus, mixer 254 may combine a harmonic of the higher frequency RF detection signal from frequency multiplier 252 with the LCHP RF signal from antenna 256. Thus, mixer 254 outputs an intermediate frequency (IF) signal to amplifier 260.

Amplifier 260 may be a low noise amplifier, which amplifies the IF signal from mixer 254. The amplified IF signal from amplifier 260 is provided to filter 262. Filter 262 may be used to remove unwanted signals outside of the desired band. Thus, filter 262 may be a band pass filter. The filtered IF signal is provided from filter 262 to mixer 264.

The third RF signal (which also has the first frequency of the RF signal from signal generator 210) from third path 276 is provided to phase splitter 258. In some embodiments, phase splitter 258 provides a zero and ninety degree phase split signal. The output of phase splitter 258 is also provided to mixer 264. Mixer 264 may be an in-phase and quadrature (I/Q) mixer. The local oscillator signal may be considered to be provided by phase splitter 258 and thus corresponds to the third RF signal from third path 276. In some embodiments, mixer 264 is a direct conversion I/Q mixer. Mixer 264 is coupled to analog to digital converter (ADC) 266. As indicated in RF sensor system 200, both the in-phase (I) and quadrature (Q) signals may be provided to ADC 266. ADC 266 digitizes the I and Q signals and provides a digitized output.

RF sensor system 200 may share the benefit(s) of RF sensor system 100. In some embodiments, transmitter subsystem 120 may utilize low power, smaller, and lower cost components for generating the output signal. For example, the frequency multipliers 222 and 226 used for up converting the RF signal from signal generator 110 may reduce the size, power consumption and cost of transmitter subsystem. Use of the RF signal from RF generator 110 for both up conversion in transmitter subsystem 120 and down conversion and detection in receiver subsystem 150 may reduce noise and increase sensitivity. Further, generation of additional local oscillator signals or other signals for transmission and/or detection may be avoided. Thus, size and power consumption of RF sensor system 200 may be reduced. In addition, the transmitted signal radiated from transmitter subsystem 220 and the LHCP signals received in receiver subsystem 250 may be digitally processed to determine the precise location of the target. In some embodiments, the frequencies used in (e.g. from RF signal paths 274, 276, and 278) may be widely separated. Thus, interstage filtering may be reduced or avoided. In other embodiments, some interstage filtering may be used, for example for impedance matching. Use of the second and third RF signals on paths 274 and 276 in demodulating the received signal may further reduce the noise. For example, phase noise degradation may be reduced. Use of RHCP antenna 228 and LHCP antenna 256, particularly in conjunction with screen 280, improve the transmit to receive isolation for non-reflected leakage paths. Two stage down conversion and detection in receiver subsystem 250 using second and third RF signals on paths 274 and 276 may allow for a high performance image reject mixer to be used in the second frequency conversion to DC, which relies on an accurate 0 and 90 degree phase generation. Moreover, if antennae 228 and 256 are parabolic and mounted such that mechanical scanning of target 202 can be performed, a high-resolution image of target 202 may be obtained. Thus, performance, cost, and/or size of RF sensor system 100 may be improved.

For example, waveform generator 212 may provide a signal at 13.61 GHz. Frequency multiplier 214 may multiply the 13.61 GHz frequency by an integer, such as 2. In such embodiments, frequency multiplier 214 outputs a 27.33 GHZ RF signal. The 27.33 GHZ signal is provided to amplifier 216, which may account for attenuation of the signal. Thus, signal generator 210 may provide a 27.33 GHZ RF signal. Splitter 270 splits the 27.33 GHz signal into three 27.33 GHZ RF signals on paths 272, 274, and 276.

Path 272 inputs the 27.33 GHZ RF signal to transmitter subsystem 220. Frequency multiplier 222 may multiply the frequency by a number, such as 3. Thus, frequency multiplier 222 may output an 81.66 GHz RF signal. The 81.66 GHz RF signal is amplified by amplifier 224. Frequency multiplier 226 multiplies the frequency of the output of amplifier 224 by a number, such as 3. Thus, frequency multiplier 226 provides a 244.98 GHz signal. Antenna 228 then outputs a 244.98 GHz RHCP RF signal. The 244.98 GHz RHCP RF signal radiates through screen 280 (which is transparent to RHCP signals) and reflects off target 202. After reflection the signal is a 244.98 GHz LHCP signal. Because the reflected signal is an LHCP signal, this 244.98 HZ LHCP signal is reflected by screen 280. Antenna 256 picks up this 244.98 GHz LHCP RF signal.

The received 244.98 GHz LHCP RF signal is provided to mixer 254. Similarly, the second 27.33 GHZ RF signal on path 274 is provided to frequency multiplier 252. Frequency multiplier 252 multiplies the frequency of the second 27.33 GHZ RF signal by a number, such as 4. Thus, frequency multiplier 252 may output a 108.88 GHz RF detection signal. The 108.88 GHz RF detection signal and the 244.98 GHz LHCP signal are provided to mixer 254. In some embodiments, mixer 254 may use the second harmonic of 108.88 GHz signal. Thus, a 27.22 GHz IF signal may be output by mixer 254 and provided to amplifier 260, which amplifies the input signal. Filter 262 may pass signals in a band of frequencies around 27.22 GHz and provide the filters 27.22 GHz LHCP signal to mixer 264.

The third 27.33 RF signal on path 276 is provided to phase splitter 258. The output is provided to I/Q mixer 264 along with the 27.22 GHz filtered RF signal. The resulting I and Q signals are provided to, for example, ADC 266.

Thus, RF sensor system 200 may share the benefit(s) of RF sensor system 100. More specifically, RF sensor system 200 may provide a low cost, low noise detection of RF signals. Thus, performance, cost, and/or size of RF sensor system 100 may be improved. Using the RF signal detected by RF sensor 200, the physiological characteristics described herein may be better measured.

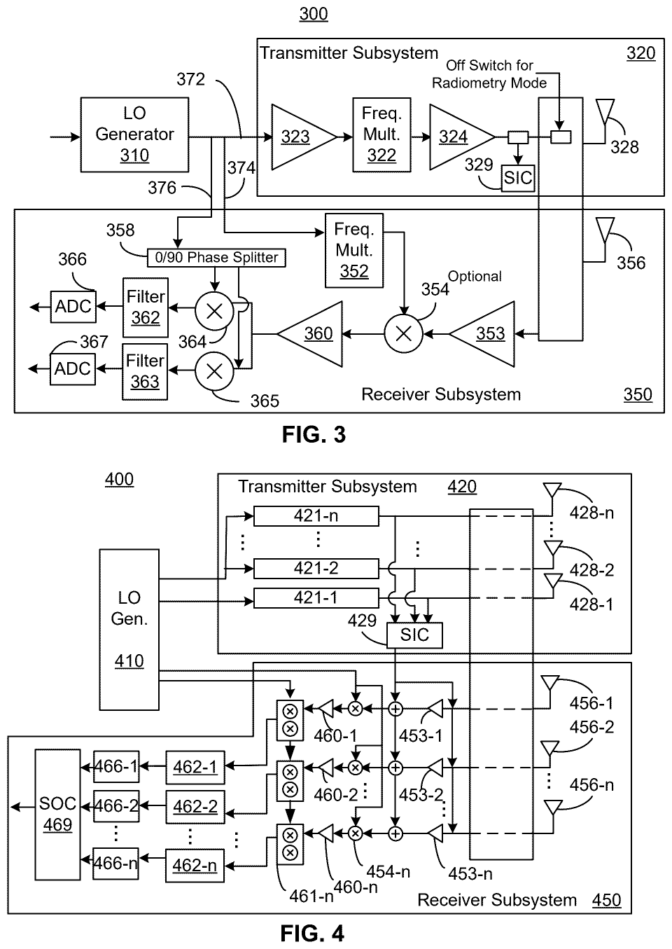

FIG. 3 is a diagram of an embodiment of RF sensor system 300. RF sensor system 300 is analogous to RF sensor system(s) 100 and/or 200. RF sensor system 300 thus may include similar components and/or have similar functions to RF sensor system 100 and/or 200. For example, RF sensor system 300 may be used in wearables and/or other devices, may measure physiological characteristics (e.g., heart rate, temperature, imaging etc.), and may share the benefits of RF sensor system 100. RF sensor system 300 thus includes signal generator 310, transmitter subsystem 320, and receiver subsystem 350 that are analogous to signal generator 110/210, transmitter subsystem 120/220, and receiver subsystem 150/250, respectively. Although not shown, RF sensor system 300 may also include a splitter and a screen analogous to splitter 270 and screen 280, respectively. For simplicity, the target is also not shown.

Thus, RF sensor system 300 includes a local oscillator (LO) generator 310 as a signal generator. In some embodiments, LO generator 310 may include a phase-locked loop (PLL) PLL and/or a digital-to-analog converter (DAC) board. LO generator 310 may be used in radar mode, radiometry mode, spectroscopy mode, or in another mode. For example, the LO generator 310 may be used in continuous wave mode for radiometry and/or spectroscopy and in frequency modulated (FM) continuous wave mode for radar.

The output signal from LO generator 310 is split into three paths 372, 274 and 376 analogous to paths 272, 274, and 276. Thus, the RF signal provided by LO generator 310 is used by transmitter subsystem 320 and receiver subsystem 350. Transmitter subsystem 320 includes amplifiers 323 and frequency multiplier 322. Frequency multiplier 322 and amplifiers 323 and 324 are analogous to frequency multiplier 222 and amplifier 224. In some embodiments, amplifier 324 may be omitted. Thus, transmitter subsystem 320 may multiply the frequency of and amplify the power for the RF signal provided on first path 372 by LO generator 310. This amplified, higher frequency RF signal is used to drive antenna 328. Also shown is optional self-interference cancellation (SIC) block 329. SIC block 329 allows the transceiver of RF sensor system 300 to transmit and receive on closely spaced frequencies by mitigating the interference between transmitter subsystem 320 and receiver subsystem 350.

Receiver subsystem includes frequency multiplier 352, mixer 354, antenna 356, phase splitter 358, amplifier 360, filters 362 and 363, and mixers 364 and 365 that are analogous to frequency multiplier 252, mixer 254, antenna 256, phase splitter 258, amplifier 260, filter 262, and mixer 264. Also shown is optional amplifier 353. Amplifiers 353 and 360 may together provide amplification analogous to that of amplifier 260.

The RF signal reflected from a target (not shown) may be sensed by antenna 356. Antenna 356 may be an LHCP antenna. The signal may be amplified and is provided to mixer 354. The RF signal from second path 374 has its frequency multiplied. In some embodiments, frequency multiplier 322 provides a multiplication of the frequency by M, while frequency multiplier 352 provides a multiplication of the frequency by a frequency N. In some embodiments M and N are (but need not be) integers. some embodiments, N=M±α, where α=1 in some embodiments. However, a may be another number. Thus, mixer 354 combines the RF signals from frequency multiplier 352 and, if used, amplifier 353. Mixer 354 provides an intermediate frequency (IF) signal to amplifier 360.

The RF signal on third path 376 is provided to phase splitter 358. Thus, I and Q signals are provided to mixers 364 and 365. Filters 362 and 363 may be band pass filters analogous to filter 262. The filtered signals are provided to ADCs 366 and 367 that are analogous to ADC 266.

RF sensor system 300 thus functions in an analogous manner to RF sensor systems 100 and 200. Thus, RF sensor system 300 may share the benefit(s) of RF sensor system(s) 100 and/or 200. More specifically, RF sensor system 300 may provide a low cost, low noise detection of RF signals. Thus, performance, cost, and/or size of RF sensor system 300 may be improved. Using the RF signal detected by RF sensor 300, the physiological characteristics described herein may be better measured.

FIG. 4 is a diagram of an embodiment of RF sensor system 400. RF sensor system 400 is analogous to RF sensor system(s) 100, 200, and/or 300. RF sensor system 400 includes multiple RF sensors (e.g. multiple transmitter-receiver pairs) and may be used as a phased array system. RF sensor system 400 thus may include similar components and/or have similar functions to RF sensor system(s) 100, 200, and/or 300. For example, RF sensor system 400 may be used in wearables and/or other devices, may measure physiological characteristics (e.g., heart rate, temperature, imaging etc.), and may share the benefits of RF sensor system 100. RF sensor system 400 includes signal generator 410, transmitter subsystem 420, and receiver subsystem 450 that are analogous to signal generator 110/210/310, transmitter subsystem 120/220/320, and receiver subsystem 150/250/350, respectively. Although not shown, RF sensor system 400 may also include a splitter and a screen analogous to splitter 270 and screen 280, respectively. For simplicity, the target is also not shown.

RF sensor system 400 includes multiple transmitters and multiple receivers. Thus, transmitter subsystem 420 includes multiple transmitter modules 421-1 through 421-n (collectively or generically 421). Each transmitter module 421 may include components analogous to transmitter subsystem(s) 220 and/or 320. For example, transmitter module 421 may include amplifiers and frequency multiplier(s) analogous to amplifiers 323 and 324 and frequency multiplier 322. A transmitter module 421 is coupled to corresponding antenna 428-1 through 428-n (collectively or generically antenna 428). Thus, RF sensor system 400 may be viewed as including multiple (i.e., n) transmitters in transmitter subsystem 420. Also shown is SIC module 429 analogous to SIC module 529.

Similarly, receiver subsystem 450 includes multiple receivers (i.e. n receivers), each of which is analogous to receiver subsystem 250 and/or 350. Receiver subsystem 450 includes antenna 456-1 through 456-n, amplifiers 453-1 through 453-n, mixers 454-1 through 454-n, amplifiers 461-1 through 462 461-n, filters 462-1 through 462-n, and ADCs 466-1 through 466-n, and SOC 469 that are analogous to antenna 356, amplifiers 353, mixers 354-1 through 354-n, amplifier 360, filters 362-1 through-n, and ADCs 366-1 through 366-n, and SOC 369.

RF sensor system 400 thus includes multiple transmitters and receivers, each of which may function in an analogous manner to RF sensor systems 100, 200, and/or 300. Thus, RF sensor system 400 may share the benefit(s) of RF sensor system(s) 100, 200, and/or 300. More specifically, RF sensor system 400 may provide a low cost, low noise detection of RF signals. Thus, performance, cost, and/or size of RF sensor system 400 may be improved. Using the RF signal detected by RF sensor 400, the physiological characteristics described herein may be better measured. Further, because RF sensor system 400 includes multiple transceivers (e.g. a transmitter and receiver) RF sensor system 400 may image a region and may operate multiple transceivers together to perform various functions. Thus, performance may be improved.

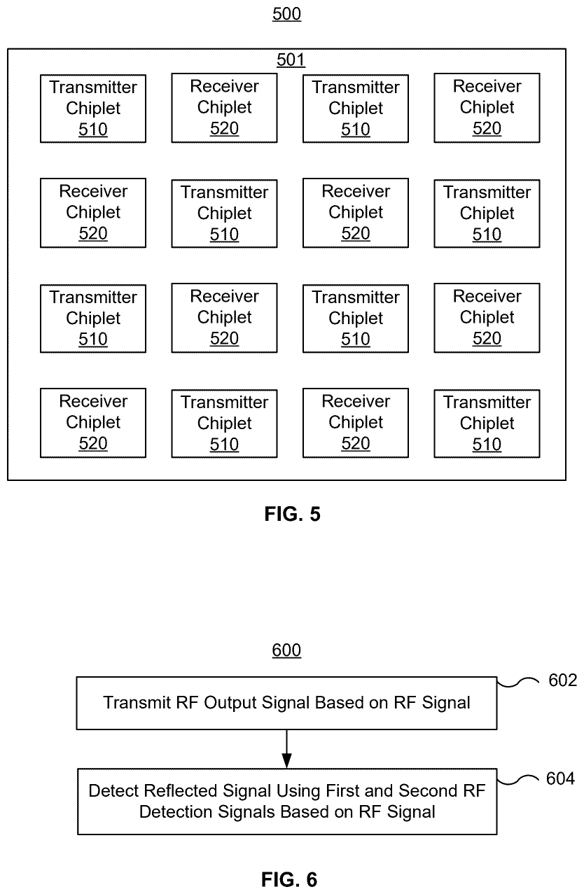

FIG. 5 is a diagram of an embodiment of an RF sensor system 500. In RF sensor system 500, transmitter subsystems and receiver subsystems are provided on separate integrated circuits (“chiplets”) 510 and 520 and arranged in an array on another semiconductor substrate or interposer 501. Thus, an array of transmitter chiplets 510 and receiver chiplets 550 is shown. Each transmitter chiplet 510 may include one or more transmitter subsystems such as transmitter subsystem 120, 220, 320, and/or 420. Each receiver chiplet 550 includes one or more receiver subsystems 150, 250, 350, and/or 450. A signal generator, such as a LO generator may also be present in die (or interposer) 501 or may be on one or more of chiplets 510 and/or 520 but is not expressly shown. In some embodiments, such a signal generator may be off chip.

For example, CMOS integrated circuits that may be used for the transmitter local oscillator (LO) generation and amplification, the receiver intermediate frequency (IF) amplification, receiver in-phase quadrature (I/Q) mixers, baseband (BB) filtering, and analog to digital converters may be present in CMOS die 501. The transmitter frequency multiplier(s), transmitter power amplifiers, and transmitter antennae may be provided in separate transmitter chiplets 520. Such chiplets 520 may include SiGe or InP to support the frequencies of greater than 200 GHz. Receiver chiplets 520 may also be formed of SiGe or InP. Receiver chiplets 520 may contain the receiver antennae, receiver low noise amplifiers, receiver mixers, and receiver IF multipliers. These two chiplets 510 and 520 may be directly attached to the CMOS die 501. The antennas may be patterned in metal on the backside of the chiplet die. Other arrangements for the circuitry may be possible.

RF sensor system 500 thus includes multiple transmitters and receivers, each of which may function in an analogous manner to RF sensor systems 100, 200, 300, and/or 400. Thus, RF sensor system 500 may share the benefit(s) of RF sensor system(s) 100, 200, 300, and/or 400.

FIG. 6 is a flow-chart depicting an embodiment of method 600 for sensing using an RF sensor system. Although described in the context of RF sensor system 100, method 600 may be used in connection with other RF sensor systems. In addition, although described in the context of particular processes, each process may include multiple substeps.

An RF output signal is provided based on an RF signal that is generated, at 602. For example, a signal generator may provide an RF signal to a transmitter subsystem. At 602, the transmitter subsystem further processes the RF signal and outputs a higher frequency RF signal via an antenna. For example, the transmitter subsystem may use frequency multipliers to increase the frequency of the RF output signal. Amplifiers may be used to account for attenuation or boost the power of the output signal for other reasons. The RF signal output is radiated toward and reflected by a target.

At 604, the RF signals reflected by the target are sensed using multiple (e.g. two or more) RF signals from the signal generator, at 604. The two RF signals may be used by a receiver subsystem to down convert the frequency of the reflected RF signal and sense the reflected RF signal in multiple stages.

For example, in RF sensor system 100, signal generator 110 may provide an RF signal or a particular frequency to transmitter subsystem 120. At 602, the transmitter susbsystem 120 may use one or more frequency multipliers to increase the frequency of the RF signal to a desired frequency. Other processing, such as power amplification may also be performed at 602. The higher frequency RF signal is also provided to an antenna at 602, which radiates the output RF signal to a target. The target reflects the output RF signal.

At 604, the reflected RF signal is received at and sensed by receiver subsystem 150. To do so, receiver subsystem 120 uses two RF signals provided by signal generator 110. The frequency of one RF signal is increased (e.g., using a frequency multiplier) and combined with the reflected RF signal. Thus, an IF signal is provided. The other RF signal is phase split and combined with the IF signal. Thus, the characteristics of target 102 may be detected.

Using method 600, the benefits of RF sensor systems 100, 200, 300, 400, and/or 500 may be achieved. For example, RF detection of physiological characteristics such as temperature, heart rate, and images of the skin may be achieved. Detection of these physiological characteristics may be made possible by the use of three RF signals from the same signal generator that provides lower noise detection. Further, the sensor systems used with method 600 may consume less power, be smaller, and have a lower cost. Thus, performance RF sensor systems may be improved.

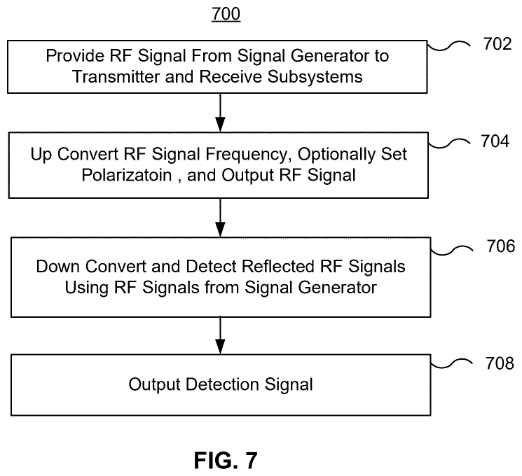

FIG. 7 is a flow-chart depicting an embodiment of method 700 for sensing using an RF sensor system. Although described in the context of RF sensor system 200, method 700 may be used in connection with other RF sensor systems, such as RF sensor systems 100, 300, 400, and/or 500. In addition, although described in the context of particular processes, each process may include multiple substeps.

An RF signal is generated and provided to transmitter and receiver subsystems, at 702. Also at 702, at least one RF signal is provided to the transmitter subsystem, while at least two signals are provided to the receiver subsystem.

At 704, the frequency of the RF signal is increased (i.e. upconverted). In some embodiments, the RF signal may also be amplified. Such amplification may account for power losses. In some embodiments, the polarization of the output signal is also controlled at 704. For example, an RHCP antenna may be used. In some embodiments, 704 is performed by a transmitter subsystem. Thus, the RF signal radiates to and is reflected by the target. The reflected signal may have a different polarization (e.g., LHCP) than the output RF signal.

At 706 the frequency of the received, reflected signal is down converted and the reflected signal otherwise processed in order to be detected. This detection process uses two RF signals provided by the RF signal generator. For example, one RF signal may have its frequency increased and be combined with the reflected RF signal to provide an IF signal. Another RF signal may be phase split and combined with the IF signal in an I/Q mixer. The resultant may be converted to digital information and output at 708.

For example, at 702, signal generator 210 may output an RF signal. Also at 702, splitter 270 splits the RF signal into three paths 272 (to transmitter subsystem), 274 (to receiver subsystem), and 276 (also to receiver subsystem).

At 704, transmitter subsystem 220 processes the RF signal for output. For example, using frequency multipliers 222 and 226 and amplifier 224 the frequency of the RF signal may be increased to the desired frequency and provided to antenna 228. Antenna 228 may be an RHCP antenna. Further, antenna 228 may be parabolic or otherwise configured to confine the output radiated RF signal. Thus, at 704, an output signal is radiated to target 202.

At 706, the LHCP reflected RF signal is received at receiver subsystem 250. The RF signal from path 274 is frequency multiplied and combined with the LHCP reflected RF signal at mixer 254. Thus, an IF signal is provided. Also at 706, the IF signal is amplified and filtered for the desired frequency band using amplifier 260 and filter 262. St 706, the RF signal on path 276 is phase split (e.g. to zero and ninety degrees). These signals are combined with the filtered IF signal from filter 262 in I/Q mixer 264. The resulting signals may be converted to digital signals by ADC 266. Thus, the reflected RF signals have been detected. The output signals are provided, at 708.

Using method 700, the benefits of RF sensor systems 100, 200, 300, 400, and/or 500 may be achieved. For example, RF detection of physiological characteristics such as temperature, heart rate, and images of the skin may be achieved. Detection of these physiological characteristics may be made possible by the use of three RF signals from the same signal generator that provides lower noise detection. Further, the sensor systems used with method 700 may consume less power, be smaller, and have a lower cost. Thus, performance RF sensor systems may be improved.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A sensor system, comprising:
a signal generator configured to provide a radio frequency (RF) signal having a first frequency;
a transmitter subsystem, coupled to the signal generator, the transmitter subsystem configured to transmit an output signal based on the RF signal, the output signal having a second frequency greater than the first frequency; and
a receiver subsystem, coupled to the signal generator, the receiver subsystem configured to detect a reflected signal from a target based on a first detection signal and a second detection signal, the first detection signal and the second detection signal being based on the RF signal.

2. The sensor system of claim 1, further comprising:
a splitter coupled to the signal generator, the transmitter subsystem, and the receiver subsystem, the splitter splitting the RF signal into a plurality of signals including a first RF signal input to the transmitter subsystem, a second RF signal, and a third RF signal, the second RF signal and the third RF signal being input to the receiver subsystem, the second RF signal corresponding to the first detection signal, and the third RF signal corresponding to the second detection signal.

3. The sensor system of claim 2, wherein the transmitter subsystem includes at least one frequency multiplier coupled with the splitter and configured to multiply the first frequency of the first RF signal to provide the second frequency of the output signal.

4. The sensor system of claim 2, wherein receiver subsystem further includes:
a multiplier coupled with the splitter and configured to multiply the first frequency of the second RF signal for the first detection signal, the first detection signal having a first detection signal frequency; and
a first mixer coupled with the multiplier and configured to mix the reflected signal with the first detection signal to provide an intermediate frequency signal having an intermediate frequency.

5. The sensor system of claim 4, wherein the receiver subsystem further includes:
a phase splitter receiving the third RF signal and outputting the second detection signal; and
a second mixer coupled with the phase splitter and the first mixer, the second mixer combining at least a portion of the intermediate frequency signal and the second detection signal, the second mixer outputting a detection signal.

6. The sensor system of claim 5, wherein the receiver subsystem further includes:
an analog-to-digital (ADC) converter coupled with the second mixer, the ADC converter providing a digital output signal based on the detection signal.

7. The sensor system of claim 5, wherein the receiver subsystem further includes:
an amplifier coupled with the first mixer; and
a band pass filter coupled with the amplifier and the second mixer, the band pass filter configured to pass a frequency range including the intermediate frequency.

8. The sensor system of claim 1, wherein the sensor system is configured to detect, based on the reflected signal, at least one of heart rate, heart rate variation, human skin, temperature, skin characteristics, blood vessels, or a change in blood vessels.

9. A wearable system, comprising:
a sensor subsystem including a signal generator, a transmitter subsystem coupled to the signal generator, and a receiver subsystem coupled to the signal generator, the signal generator being configured to provide a radio frequency (RF) signal having a first frequency, the transmitter subsystem being configured to transmit an output signal based on the RF signal, the output signal having a second frequency greater than the first frequency, the receiver subsystem being configured to detect a reflected signal from a target based on a first detection signal and a second detection signal, the first detection signal and the second detection signal being based on the RF signal; and
an attachment subsystem configured to couple the sensor subsystem to a user.

10. The wearable system of claim 9, wherein the sensor subsystem further includes:
a splitter coupled to the signal generator, the transmitter subsystem, and the receiver subsystem, the splitter splitting the RF signal into a plurality of signals including a first RF signal input to the transmitter subsystem, a second RF signal, and a third RF signal, the second RF signal and the third RF signal being input to the receiver subsystem, the second RF signal corresponding to the first detection signal, and the third RF signal corresponding to the second detection signal.

11. The wearable system of claim 9, wherein the sensor subsystem is incorporated into at least one of a watch or a smart phone.

12. The wearable system of claim 10, wherein the attachment subsystem includes at least one of a wrist band or a chest band.

13. A method, comprising:
transmitting, by a transmitter subsystem coupled to a signal generator that generates a radio frequency (RF) signal having a first frequency, an output signal, the output signal being based on the RF signal, the output signal having a second frequency greater than the first frequency; and
detecting, by a receiver subsystem, a reflected signal from a target, the detecting being based on a first detection signal and a second detection signal, the first detection signal and the second detection signal being based on the RF signal.

14. The method of claim 13, further comprising:
splitting the RF signal into a plurality of signals including a first RF signal input to the transmitter subsystem, a second RF signal, and a third RF signal, the second RF signal and the third RF signal being input to the receiver subsystem, the second RF signal corresponding to the first detection signal, and the third RF signal corresponding to the second detection signal.

15. The method of claim 14, wherein the transmitting further comprises:
multiplying the first frequency of the first RF signal to provide the second frequency of the output signal.

16. The method of claim 14, wherein the receiving further includes:
multiplying the first frequency of the second RF signal for the first detection signal, the first detection signal having a first detection signal frequency; and
mixing the reflected signal with the first detection signal to provide an intermediate frequency signal having an intermediate frequency.

17. The method of claim 16, wherein the receiving further includes:
receiving the third RF signal at a phase splitter and outputting, by the phase splitter, the second detection signal; and
mixing at least a portion of the intermediate frequency signal and the second detection signal, thereby providing a detection signal.

18. The method of claim 17, wherein the receiving further includes:
analog-to-digital (ADC) converting the detection signal to provide a digital output signal.

19. The method of claim 17, wherein the receiving further includes:
amplifying the intermediate frequency signal to provide an amplified intermediate; and
filtering the amplified intermediate frequency signal to pass a frequency range including the intermediate frequency, thereby providing the at least the portion of the intermediate frequency signal for mixing with the second detection signal.

20. The method of claim 13, wherein the detecting is configured to detect, based on the reflected signal, at least one of heart rate, heart rate variation, human skin, temperature, skin characteristics, blood vessels, or a change in blood vessels.
